(19) United States
(12) Patent Application Publication
(10) Pub. No.: US 2026/0174746 A1
(43) Pub. Date: Jun. 25, 2026

(54) Pendulum Mounting for Machines for Processing Workpieces of Wood, Plastic, or the Like, and Machine Having at Least One Such Pendulum Mounting

(71) Applicant: Michael Weinig AG, Tauberbischofsheim (DE)

(72) Inventors: Lucas BLESCH, Ahorn-Eubigheim (DE); Benjamin FRANK, Boxberg (DE); Andreas KUHN, Kitzingen (DE); Ralf WAGNER, Hardheim (DE)

(73) Assignee: Michael Weinig AG, Tauberbischofsheim (DE)

(21) Appl. No.: 19/420,965

(22) Filed: Dec. 16, 2025

(30) Foreign Application Priority Data
Dec. 18, 2024 (DE) 102024004400.0

(51) Int. Cl. A61K 31/4745

(52) U.S. Cl. CPC A61K 31/4745

(57) ABSTRACT

A pendulum device for machines for processing workpieces made of wood, plastic, and the like includes at least one feed roller rotatably mounted on a pendulum. At least one actuator is provided on the pendulum, the actuator having a cylinder in which a piston is displaceable. A piston rod of the piston is connected to the pendulum and is pressure-loaded such that an oscillating movement of the feed roller is brought to an end within a very short time in a structurally simple manner. The piston separates an air chamber from an oil chamber, the oil chamber being conductively connected to a further oil chamber of a pressure transducer. A hydraulic medium located in the oil chamber of the pressure transducer is under pressure.

### CROSS-REFERENCE TO RELATED APPLICATION

This application claims the benefit of German Patent Application No. DE 10 2024 004 400.0, filed 18 Dec. 2024, the content of which is incorporated by reference in its entirety.

### TECHNICAL FIELD

The disclosure relates to a pendulum device for machines for processing workpieces made of wood, plastic and the like, and to a machine with at least one such pendulum device.

### BACKGROUND

In processing machines, in particular woodworking machines, in particular moulding machines, the workpieces are guided through the machine with the aid of feed rollers. The feed rollers are rotatably mounted in pendulums, on each of which is provided an actuator. The actuator has a pneumatic cylinder and a mechanism by means of which the feed rollers are set in a home position so that the workpieces to be processed lift the feed rollers as they are transported through the machine. The feed roller is loaded in the direction of the workpiece via the pneumatic cylinder which is charged with a pressure medium. Since the workpieces are transported at high speed, the feed rollers are deflected abruptly upwards and flung into the air. As a result of being flung upwards, they first lift off the workpiece and then fall back onto it. This occurs as an elastic impact, as it were, causing the feed roller to be flung upwards again and lift off the workpiece once more. This process repeats several times until the feed rollers rest quietly on the workpiece. This means that the oscillating or pendulum movement of the feed rollers gradually subsides, allowing the feed rollers to rest on the workpieces and transport them. The oscillating movement of the feed rollers only comes to an end after the workpieces have travelled a relatively long distance.

This repeated lifting from and landing back on the workpiece, on the one hand, negatively impacts the transportation of the workpieces. On the other, the workpieces are no longer held cleanly at the moment of lift-off and are caused to oscillate when the feed roller strikes the workpiece, which in both cases negatively affects the surface quality during processing.

### SUMMARY

The disclosure is based on the object of designing the generic pendulum device and the machine so that the oscillating movement of the feed rollers is brought to an end in a structurally simple manner within a very short time.

This object is achieved with the pendulum device as disclosed and claimed.

In the pendulum device, the cylinder piston is provided so that it lies between the air chamber and the oil chamber. When the feed roller is lifted by the workpiece, the piston moves towards the oil chamber, which is conductively connected to the oil chamber of the pressure transducer. The upwards movement of the piston displaces the hydraulic medium from the cylinder's oil chamber into the pressure transducer's oil chamber. The displaced hydraulic medium does not enter the pressure transducer abruptly, but rather, due to a throttling effect of the conductive connection between the two oil chambers, only at a limited flow velocity. This results in a very high damping effect, which means that the feed roller is no longer thrown upwards as violently upon contact with the workpiece, and the oscillation/pendulum movement subsides within a very short time. This significantly improves the transportation of workpieces through the machine. The pendulum's oscillation is not transmitted to the workpiece, thus creating a very smooth run. This, in turn, is crucial for the quality of the workpiece processing.

Not only are oils or hydraulic oils suitable as the hydraulic medium, but also other hydraulic fluids, such as water-based hydraulic fluids. Therefore, when reference is made to oil chambers, this should not be understood as a limitation to oils.

The hydraulic medium located in the oil chamber of the pressure transducer is preferably under pressure. This exerts a force on the feed pendulum, which can be adjusted by changing the pressure.

Gas, in particular compressed air, is advantageously used as the pressure medium. Compressed air is readily available. It is an easy-to-handle medium.

The provision of a throttle point for the hydraulic medium in the conductive connection between the oil chamber of the cylinder and the oil chamber of the pressure transducer advantageously contributes to achieving the desired damping characteristics. The throttle point ensures that the hydraulic medium can only be displaced to a limited extent, which is beneficial for the damping effect.

The pendulum device advantageously has two feed rollers, the pendulums of which are articulated and connected in each case to an actuator.

In this case, it is particularly advantageous if both cylinders of the two actuators are connected to a common pressure transducer. This results in a structurally very simple design for the pendulum device.

It is possible, for example, for two cylinders to act on a pendulum, meaning that, when there are two pendulums, four cylinders are connected to the pressure transducer.

It is advantageous if the conductive connection between the oil chambers of the actuator and the pressure transducer has a hose. This hose is flexible and allows the conductive connection between the two units regardless of their installation location in the pendulum device.

In a particularly advantageous embodiment, the throttle point is formed by the connection of the hose to the cylinder.

The machine is characterized by being equipped with at least one pendulum device. The machine therefore enables optimal transportation guidance of the workpieces, so that no quality limitations occur on the workpiece surface.

A particularly advantageous embodiment of the machine is a moulding machine with which the workpieces can be processed on all four sides as they pass through.

The damped feed pendulum is advantageously used in the in-feed of the moulding machine. Here, variations in the raw wood can cause the pendulum to deflect particularly sharply depending on the transportation speed. Ultimately, the relationship between excess raw wood and transportation speed is crucial. However, it may also be advantageous to use damped feed pendulums within the machine where the workpieces have already been partially processed, e.g. before each processing spindle.

### BRIEF DESCRIPTION OF THE DRAWINGS

The invention is explained in more detail with reference to an exemplary embodiment illustrated in the drawings, in which:

FIG. 1 shows a perspective view of part of a processing machine with a pendulum device,

FIG. 2 shows a front view of the pendulum device according to FIG. 1,

FIG. 3 shows a perspective view of the pendulum device according to FIG. 2,

FIG. 4 shows a pendulum path-workpiece path diagram.

### DETAILED DESCRIPTION

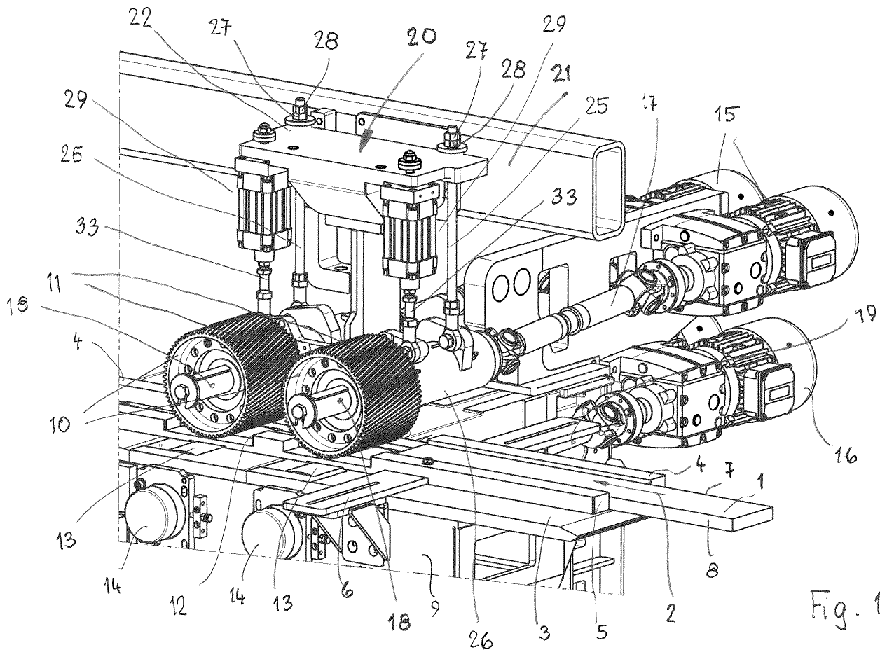

The processing machine serves for processing workpieces made of wood or wood-like materials. The workpieces 1 are in the form of boards and have, for example, a rectangular cross section (FIG. 1). The workpieces 1 are transported through the processing machine in their longitudinal direction in the direction of arrow 2. The workpieces 1 are processed in a suitable manner as they pass through the machine.

In this exemplary embodiment, the processing machine is a moulding machine with which the workpieces 1 are processed on all four sides as they pass through the machine.

The workpieces 1 to be processed are fed on an in-feed table 3 which can consist of individual table plates.

Located on the in-feed table 3 are a right-hand stop guide 4 and a left-hand stop guide 5 in the transportation direction 2. The two stop guides 4 and 5 extend parallel to one other in the transportation direction 2.

The left-hand stop guide 5 can be adjusted perpendicularly to the transportation direction 2 to accommodate workpieces 1 of different widths. The corresponding adjustment mechanism 6 is shown schematically in FIG. 1.

As they are transported through the machine, the workpieces rest with their parallel longitudinal sides 7, 8 against the stop guides 4, 5, which guide the workpiece 1 precisely as it passes through the machine. This allows the workpiece 1 to be processed cleanly by corresponding tools.

The in-feed table 3 with the stop guides 4, 5 and the adjustment mechanism 6 is located on a stand 9 which is mounted so as to be height-adjustable on a machine stand (not shown) for setting a chip removal on a first lower tool (also not shown).

Feed rollers 10 serve to transport the workpieces 1 through the machine, these rollers lying spaced apart one behind the other in the transportation direction 2 within the machine and bearing against the workpieces 1 under force. The feed rollers 10 are rotatably driven about their axis and advantageously have a knurling or toothing 11 on their outer surface which ensures reliable transportation of the workpieces 1 by the feed rollers 10.

At the level of the feed rollers 10, in the area below a transportation track 12 for the workpieces 1, there are located table rollers 13 rotatable about horizontal axes, these table rollers bearing against the underside of the workpieces 1 as they pass through the machine and contributing to the feeding of the workpieces 1 through the machine.

The axes of the feed rollers 10 and the table rollers 13 run horizontally and perpendicularly to the transportation direction 2.

Drive motors 15 and 16 are provided to drive the feed rollers 10 and the table rollers 13, the motors being suitably fastened to the machine stand. The drive motors 15 are connected for drive purposes via cardan shafts 17 to shafts 18 on which the feed rollers 10 are rotationally fixedly seated.

The drive motors 16 are also connected for drive purposes via cardan shafts 19 to shafts (not shown) on which the table rollers 13 are rotationally fixedly mounted.

The cardan shafts 17, 19 allow the height of the feed rollers 10 and the table rollers 13 to be adjusted to accommodate workpieces 1 of varying thickness. The table rollers 13 are mounted in the stand 9 of the in-feed table 3 and are each provided with a cover 14 facing the front of the machine.

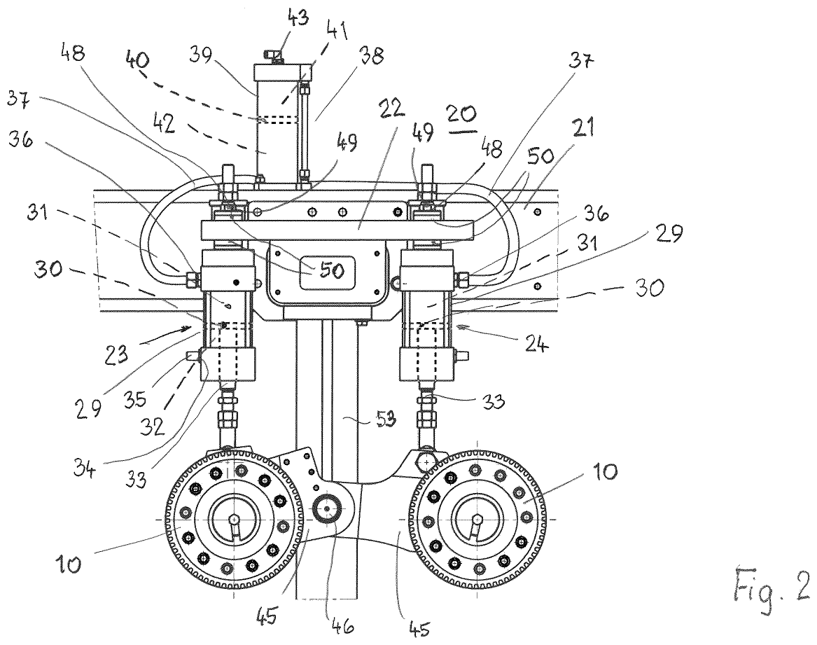

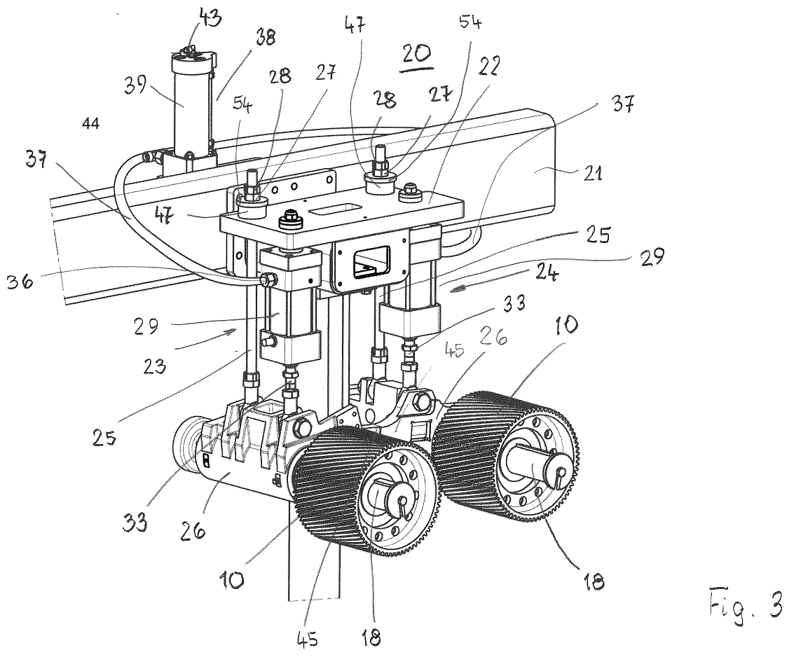

The feed rollers 10 are part of a pendulum device 20, which is shown in FIGS. 2 and 3. For clarity, only a portion of the pendulum device 20 is shown in FIG. 1. The pendulum device 20 is suspended from a transportation beam 21, which is located at a distance above the transportation track 12 and extends in the transportation direction 2 of the workpieces 1. A bracket 22 is fastened to the transportation beam 21, projecting transversely from the transportation beam 21, and two actuators 23 and 24 are mounted thereon, these allowing the feed rollers 10 to be adjusted vertically and subjected to a force. Since both actuators 23 and 24 are designed to be identical, the actuator 23 will be described in more detail below.

It has an actuating rod 25, the upper end of which projects through a bore in the plate-shaped bracket 22, and the lower end of which is articulated to a pendulum 26. The pendulum 26 forms a bearing for the shaft 18 of the feed roller 10.

The actuating rod 25 is mounted so as to be displaceable in a known manner in the bracket 22 so that the distance of the pendulum 26, and thus the feed roller 10, from the transportation track 12 can be adjusted by corresponding axial adjustment of the actuating rod 25 relative to the bracket 22. To axially secure the actuating rod 25, a nut 27 and a lock nut 28 are provided, these being seated on the end of the actuating rod 25 that projects beyond the bracket 22.

The actuating rod 25 is used to set the home position of the pendulum 26 and thus of the feed rollers 10.

Next to the actuating rod 25 is located a cylinder 29, which is held on the underside of the bracket 22 facing the feed rollers 10 and projects vertically downwards from it. Accommodated in the cylinder 29 is a piston 30 which separates an oil chamber 31 from an air chamber 32.

The piston 30 is connected to a piston rod 33, which extends through the air chamber 32 and protrudes through the base of the cylinder 29 in a sealed manner. The piston rod 33 is articulated to the pendulum 26.

An opening 34 leads into the air chamber 32, through which air can flow from the air chamber 32 to the outside and outside air can flow into the air chamber 32. Advantageously connected to the opening 34 is a silencer 35 which projects away from the cylinder 29 and ensures that no disturbing noises occur when the air passes through the opening 34.

The oil chamber 31 of the cylinder 29 advantageously contains hydraulic oil which serves as a damping medium for the pendulum movement of the feed rollers 10 upon contact with the workpieces 1, as will be described later.

A connection 36 opens into the oil chamber 31 and is conductively connected to a pressure transducer 38 via an oil hose 37. It is fastened to the transportation beam 21 and has a housing 39 in which a damping disc 40 is accommodated. It separates a compressed air chamber 41 from an oil chamber 42. It is connected to the oil chamber 31 of cylinder 29 via the oil hose 37.

The housing 39 has a compressed air connection 43 to which a compressed air hose can be connected. It connects the compressed air chamber 41 to a compressed air source (not shown).

The connection 36 acts as a throttle point which determines the degree of damping of the oscillating movement of the feed rollers 10.

The pressure transducer 38 is connected, as described, via the oil hoses 37 to both cylinders 29 of the actuators 23, 24 of the pendulum device 20.

The machine has several pendulum devices 20 along its length, these being configured as described. Each pendulum device 20 has two feed rollers 10 lying parallel to one other, which are seated on the shafts 18 of the pendulum 26. The two pendulums 26 have mutually directed arms 45. The free ends of the arms 45 of the pendulum 26 are articulated in a transportation slide 53 via an axle 46. The axle 46 runs parallel to the shafts 18 in the area between the two feed rollers 10 (FIG. 2).

The lower ends of the actuating rods 25 and the piston rods 33 engage each of the two pendulums 26 in an articulated manner. Discs 54 and damping discs 47 are arranged between the nuts 27 of the actuating rods 25 and the top of the bracket 22. The damping discs 47 surround the end of the actuating rods 25 that projects upwards beyond the bracket 22 and are displaceable relative to the damping disc 47. In the initial position, the nuts 27 of the actuating rods 25 rest on the damping discs 47 under the weight of the pendulum 26 with the feed rollers 10, with the disc 54 positioned in between. Additionally, the cylinder force, set via the pneumatic pressure, acts in the same direction.

The cylinders 29 have an axially projecting bolt 48 on their upper side which passes through the brackets 22 and on whose free end a nut 49 is seated. The cylinder 29 is attached to the bracket 22 with damping discs 50 in between. The damping discs 50 surround the bolt 48.

The actuating rods 25 and the cylinders 29 with the piston rods 33 are each spaced apart one behind the other and are in each case articulated to the pendulums 26 as described.

The feed rollers 10 are set with the aid of the actuating rods 25 to a home position which depends on the thickness tolerance of the raw workpieces 1 to be transported by the machine. The home position is selected so that, upon contact with the transportation rollers 10, the workpieces 1 lift them from the home position. This results in the actuating rods 25 being axially displaced relative to the bracket 22 via the pendulum 26, causing the nuts 27 on the actuating rods 25 to lift off the damping discs 47. At the same time, the piston rods 33 are also displaced relative to the cylinders 29. As described, the two feed rollers 10 are articulated via the axle 46 and are pivoted relative to one other about the axle 46 when the workpiece 1 successively reaches the feed rollers 10 during transportation through the machines. This ensures that the feed rollers always rest on the workpiece 1 and reliably transport it through the machine.

As soon as the workpieces 1, with their leading end in the transportation direction 2, contact the first feed roller 10, it is deflected upwards.

Depending on the excess raw wood and since the workpieces 1 are transported through the machines at a relatively high speed, the feed rollers 10 are not moved continuously upwards, but rather flung upwards. This results in the feed rollers 10 performing a kind of pendulum movement.

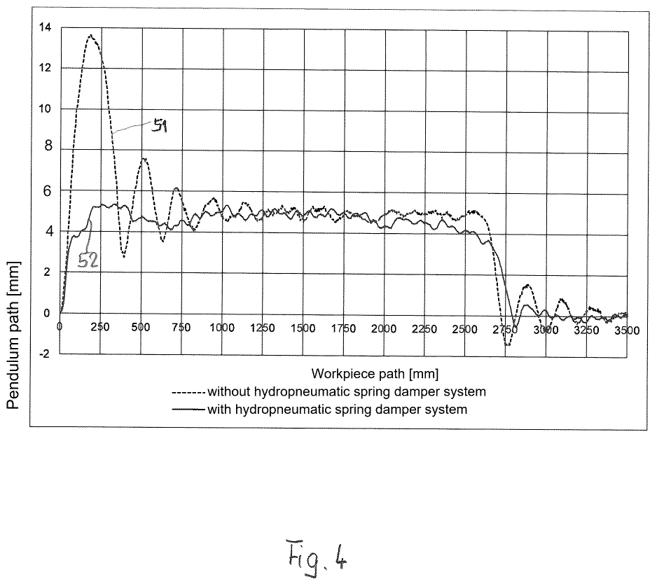

FIG. 4 shows the pendulum behaviour of the feed rollers 10. The pendulum swing is shown against the workpiece path. Curve 51 shows the pendulum behaviour of the feed rollers in conventional machines without damping. A very high pendulum swing can be seen when the workpiece 1 strikes the feed rollers 10. Further decreasing pendulum swings can be seen until the feed roller 10 is resting quietly on the workpiece 1.

As soon as the workpiece 1 has passed the feed roller 10, the feed roller drops abruptly downwards towards its home position. Considerable pendulum swings also occur here until the feed roller can assume its home position defined by the actuating rod 25.

In contrast, curve 52 shows the pendulum behaviour of the embodiment. The pendulum swings are significantly smaller both when the workpiece 1 strikes the feed roller and when it falls after passing the workpiece 1. This very high damping is achieved by the described design of the cylinder 29 in conjunction with the pressure transducer 38.

As soon as the workpiece 1 strikes the feed roller 10 on the right in FIG. 2, it is lifted upwards from its home position defined by the actuating rod 25. This results in the piston 30 being displaced upwards within the cylinder 29 via the piston rod 33. In this process, the oil located in the oil chamber 31 is displaced via the connection 36 into the oil hose 37 and from there into the oil chamber 42 of the pressure transducer 38. The oil hose connection 36 forms a throttle point when the oil is displaced from the oil chamber 31 so that the oil in the oil chamber 31 can only be displaced into the oil chamber 42 of the pressure transducer 38 with an ideal flow velocity limited for this application. The connection 36 represents the main throttle point. In addition, the connection 44 of the pressure transducer 38 and the oil hose 37 also have a throttling effect.

In conjunction with the viscosity of the hydraulic oil used, these throttle points in the hydraulic circuit result in good damping characteristics (see curve 52 in FIG. 4). The damping disc 40 rests on the oil and prevents foaming in the oil and sloshing of the oil displaced in the oil chamber 42.

The compressed air located in the compressed air chamber 41 keeps the oil located in the oil chamber 42 at the set pressure at all times. The pneumatic pressure allows the feed rollers' contact force to be optimally specified depending on the workpieces 1 being processed. Furthermore, the compressed air ensures a rapid return of the pendulum to the home position.

To keep the oil in oil chamber 42 of the pressure transducer 38 under constant pressure, at least one compression spring located in the pressure chamber 41 can also be used instead of the compressed air, for example, this spring pressurizing the oil in the oil chamber 42 in a suitable manner.

When the feed roller 10 is lifted, the piston 30 is moved upwards in the cylinder 29. Outside air is drawn in via the connection 34.

Conversely, when the piston 30 is lowered, the air located in the air chamber 32 is displaced to the outside through the connection 34. Similarly, oil is supplied from the oil chamber 42 of the pressure transducer 38 via the connection 36 and the oil hose 37. The hydropneumatic system ensures that the pendulum movements of the feed roller 10 are dampened to a very large extent as described. The good damping properties result from the fact that a hydraulic medium is directed from a cylinder-side oil chamber 31 into an oil chamber 42 of a pressure transducer 38 via throttle points in the hydraulic circuit. In this process, the kinetic energy of the hydraulic medium is converted into thermal energy. The degree of damping is determined by the cylinder connections or fittings 36, 44, which act as throttle points, and the selected diameters and lengths of the hydraulic hoses 37, in conjunction with the oil viscosity of the hydraulic medium. The desired damping can be specified using these parameters. As curve 52 in FIG. 4 shows, the swing movements of the feed roller 10 are very small once the workpiece 1 has been transported under the transportation roller 10.

The cylinder 29 is a hydraulic cylinder, preferably a low-pressure hydraulic cylinder. It has a special seal which reliably separates and seals the oil chamber from the air chamber.

1.-10. (canceled)

11. A pendulum device for machines for processing workpieces, comprising:
a feed roller (10) rotatably mounted on a pendulum (26);
a cylinder (29) provided on the pendulum (26); and
a piston (30) displaceably arranged in the cylinder (29),
wherein the piston (30) separates an air chamber (32) from an oil chamber (31),
wherein the oil chamber (31) is conductively connected to a further oil chamber (42) of a pressure transducer (38), and
wherein the further oil chamber (42) contains a hydraulic medium.

12. The pendulum device according to claim 11,
wherein the hydraulic medium located in the further oil chamber (42) of the pressure transducer (38) is pressurized.

13. The pendulum device according to claim 11,
wherein compressed air is used as a pressure medium in a pressure chamber (41) of the pressure transducer (38).

14. The pendulum device according to claim 11,
further comprising a throttle point (36, 37, 44) for the hydraulic medium conductively connected between the oil chamber (31) and the further oil chamber (43).

15. The pendulum device according to claim 11,
further comprising a further feed roller (10) rotatably mounted on a further pendulum (26),
wherein the pendulum (26) and the further pendulum (26) are articulated and each connected to a respective actuator (23, 24).

16. The pendulum device according to claim 15,
wherein the respective actuator (24) connected to the further feed roller (10) includes a further cylinder (29), and
wherein the further cylinder (29) is connected to the pressure transducer (38).

17. The pendulum device according to claim 11,
wherein the oil chamber (31) is conductively connected to the further oil chamber (42) by a hose (37).

18. The pendulum device according to claim 17,
wherein a connection to the cylinder (29) for the hose (37) is a throttle point (36).

19. The pendulum device according to claim 11,
configured for processing workpieces made of wood or plastic.

20. A machine comprising at least one pendulum device according to claim 11.

21. The machine according to claim 20, wherein the machine is a moulding machine.
